(19) United States
(12) Patent Application Publication
(10) Pub. No.: US 2008/0214568 A1
(43) Pub. Date: Sep. 4, 2008

(54) Pharmaceutical Combination Comprising an Antifungal Agent and an Active Substance Selected From Carveol, Eugenol, Thymol, Borneol, Carvacrol, and Alpha- and Beta-Ionones

(75) Inventor: Adnane Remmal, Fes (MA)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ADVANCED SCIENTIFIC DEVELOPMENTS, Casablanca (MA)

(21) Appl. No.: 11/914,005

(22) PCT Filed: May 15, 2006
(86) PCT No.: PCT/IB2006/001329

§ 371 Date: Jan. 22, 2008

(30) Foreign Application Priority Data
May 13, 2005 (IB) PCT/IB2005/001317

(51) Int. Cl. A61K 31/496; A61K 31/395; A61K 31/4196; A61P 31/10; A61K 31/513; A61K 31/505

(52) U.S. Cl. 514/254.07; 514/183; 514/383; 514/274; 514/256

(57) ABSTRACT

The present invention relates to a pharmaceutical composition. The inventive pharmaceutical composition comprises: at least a first therapeutically-active substance which is selected from carveol, thymol, eugenol, borneol, carvacrol, alpha-ionone, beta-ionone and the isomers, derivatives and mixtures thereof; and at least a second therapeutically-active substance which is an antifungal agent.

The invention relates to a pharmaceutical composition comprising two therapeutically active substances one of which exerts a potentiating action on the other, and to the use of said composition.

It is known that the efficacy of therapeutic agents depends on the doses used which, in the case of partial resistance, necessitates increasing the doses of the therapeutic agents in order to attain the desired efficacy. This dose increase leads to problems with adverse effects and acute or chronic toxicity, which may considerably complicate the condition of the treated patients.

Said partial resistance may turn into complete resistance. In this case, increasing the dose no longer has any beneficial therapeutic effect; only the toxic effects are observed. The treatment in such a case consists in changing the therapeutic agent.

This chain of events can repeat itself and lead to the most serious situation: complete resistance to multiple therapeutic agents (multidrug resistance).

For instance, in particular, immunosuppressed patients become increasingly difficult to treat and their life expectancy is correspondingly shortened. Moreover, their quality of life is substantially affected by the administration of high doses of therapeutic agents.

The invention is directed at alleviating these problems by proposing to combine at least two therapeutically active substances, one of which potentiates the activity of the other, which not only makes it possible to lower the doses of each therapeutically active substance but also to treat patients afflicted with infections caused by resistant microorganisms.

In this regard, the invention provides a pharmaceutical composition characterized in that it comprises:
at least one first therapeutically active substance selected from the group consisting of carveol, thymol, eugenol, borneol, carvacrol, alpha-ionone, beta-ionone, and the isomers, derivatives and mixtures thereof,

and,
at least one second therapeutically active substance which is an antifungal agent.

The first therapeutic substance can be obtained by chemical synthesis or from a plant source.

Preferably, the antifungal agent in the composition of the invention is selected from the group consisting of the polyenes, the imidazoles, the triazoles, the nucleoside analogues, the allylamines, the echinocandins, the sordarins, the morpholines, griseofulvin, cyclopiroxolamine, selenium sulfide, and the mixtures thereof.

More preferably, the antifungal agent is selected from the group consisting of nystatin, amphotericin B, ketoconazole, econazole, miconazole, clotrimazole, fluconazole, itraconazole, voriconazole, posaconazole, 5-fluorocytosine, naftafin, terbinafin, caspofungin, amorolfin, and the derivatives and mixtures thereof.

A more particularly preferred antifungal composition is a composition in which the first therapeutically active substance is carvacrol or eugenol, and the antifungal agent is fluconazole.

Another more particularly preferred antifungal composition is a composition in which the first therapeutically active substance is carvacrol or eugenol, and the antifungal agent is selected from voriconazole, caspofungin, itraconazole, 5-fluorocytosine, and the mixtures thereof.

The invention also proposes a kit characterized in that it comprises at least one first container containing a first therapeutically active substance selected from the group consisting of carveol, thymol, eugenol, borneol, carvacrol, alpha-ionone, beta-ionone, and the isomers and derivatives and mixtures thereof, and at least one second container containing a second therapeutically active substance which is an antifungal agent.

Lastly the invention proposes a method for treating an infection due to a fungus characterized in that one administers simultaneously or sequentially to a patient having a fungal infection, at least one first therapeutically active substance selected from the group consisting of carveol, thymol, eugenol, borneol, carvacrol, alpha-ionone, beta-ionone, and the isomers and derivatives and mixtures thereof, and at least one second therapeutically active substance which is an antifungal agent.

Preferably, in said method, one simultaneously or sequentially administers to a patient having a fungal infection between 10 and 200 mg/kg of body weight/day of said first therapeutically active substance, and between 2 and 100 mg/kg of body weight/day of said second therapeutically active substance which is an antifungal agent.

Preferably, in this method, said first therapeutically active substance is selected from the group consisting of carvacrol, eugenol and thymol and said second therapeutically active substance is selected from the group consisting of fluconazole, voriconazole, itraconazole, 5-fluorocytosine and caspofungin, and the mixtures thereof.

The invention will be better understood and other aims and advantages thereof will appear more clearly in the explanatory description which follows and which refers to the figures wherein:

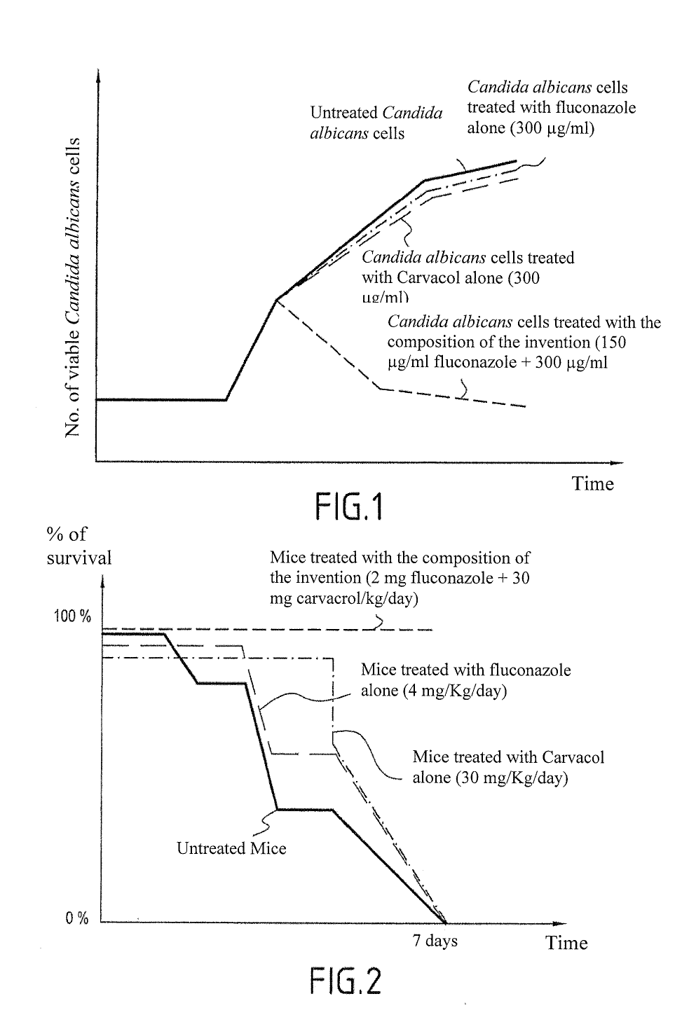

FIG. 1 presents kinetic test results on a C. albicans culture of the fungicidal action of fluconazole alone and carvacrol alone as compared with a pharmaceutical composition according to the invention comprising fluconazole and carvacrol, and

FIG. 2 presents the results of in vivo tests in a systemic candidiasis model in experimentally infected mice either untreated, or treated with fluconazole alone, treated with carvacrol alone, or treated with a pharmaceutical composition according to the invention comprising fluconazole and carvacrol.

The pharmaceutical composition according to the invention comprises as first therapeutically active substance thymol, eugenol, carvacrol, borneol, carveol, alpha-ionone, beta-ionone, and the derivatives and isomers as well as any mixtures thereof.

Said compounds have well-known antifungal properties and must be pure.

Thymol, eugenol, carvacrol, borneol, carveol, alpha-ionone, beta-ionone are found in various proportions in different aromatic plant extracts, that is to say, they can be purified from such plants. However, they can quite simply be obtained by chemical synthesis.

As a matter of fact, the inventors have now discovered that said compounds have a potentiating effect on many therapeutically active substances including known antifungal agents which are already used as medicaments specific in this field.

The second therapeutically active substance comprised in the pharmaceutical composition of the invention is therefore an antifungal agent, which is already known as such and already used as medicament specific in this field and whose activity is potentiated.

Examples of known antifungal agents already used as medicaments specific in this field which can be used in the pharmaceutical composition of the invention, and whose effect will be potentiated by the first pure therapeutically active substance, belong to three families: the pyrimidine family represented by 5-fluorocytosine, the azole family represented by fluconazole, voriconazole, itraconazole and the echinocandin family represented by caspofungin.

Said compounds can be used alone, or in combination with each other. The derivatives thereof, if they have antifungal activity, can also be used.

Particularly preferred are fluconazole, voriconazole, capsofungin and 5-fluorocytosine used in combination more particularly with carvacrol, eugenol and/or thymol.

Of course, the pharmaceutical composition according to the invention is not restricted to the use of only those antifungal agents mentioned above. In fact, considering the potentiating effect exerted by the first therapeutically active substance defined in the invention, other known or future antifungal agents can also be successfully used.

The pharmaceutical composition according to the invention can be formulated so as to be suitable for a simultaneous or sequential administration of said at least first and second therapeutically active substances.

The pharmaceutical form of the pharmaceutical composition of the invention shall be adapted to its use. For example, it can be used in the form of a solution, suspension, tablet or others. The compositions for parenteral administration are generally pharmaceutically acceptable sterile solutions or suspensions which can optionally be prepared immediately before use.

For the preparation of nonaqueous solutions or suspensions, it is possible to use natural vegetable oils like olive oil, sesame oil or paraffin oil or the injectable organic esters such as ethyl oleate. The sterile aqueous solutions can be composed of a solution of therapeutically active substances in water. The aqueous solutions are suitable for intravenous administration in so far as the pH is properly adjusted and they are made isotonic, for example by adding a sufficient amount of sodium chloride or glucose.

In fact, considering the chemical structure of antifungal agents, and secondly, considering the chemical structure of carveol, carvacrol, thymol, eugenol, borneol, alpha-ionone and beta-ionone, it is thought, without intending to be bound by this theory, that carveol, carvacrol, thymol, eugenol, borneol, alpha-ionone and beta-ionone and the isomers, derivatives and mixtures thereof, interact with the antifungal agents to form complexes having a structure which diffuses more easily into the body's physiological fluids and which diffuses more easily into the cytoplasm of target infected cells.

However, it has been shown that when the different components of the pharmaceutical composition of the invention are mixed in the presence of detergents such as Tween or Triton or solvents such as ethanol or DMSO (dimethyl sulfoxide), the active molecules of the first and second therapeutically active substance associate with the molecules of the detergents and solvents and do not form potentiating complexes.

Now it has been discovered that the potentiating complex forms when an aqueous agar suspension is used, as means of dispersion by viscosity.

Thus, the pharmaceutical composition of the invention will preferably be prepared without detergent and without solvent. For example, it will be prepared as an aqueous suspension made viscous by the addition of agar at a non-solidifying concentration, for example from 1 to 5 grams of agar per liter of suspension.

The pharmaceutical composition of the invention enables the treatment of local or systemic infections caused by resistant microorganisms using doses of each of said first and second therapeutically active substance which are lower than the doses required for treating the same infections due to susceptible microorganisms with one or the other of these same said first and second therapeutically active substances alone. In fact, the composition of the invention enables the use of doses of said first therapeutically active substance, when it is combined with said second therapeutically active substance, which are approximately three to ten times lower than the doses required when said first therapeutically active substance is used alone, and of doses of said second therapeutically active substance, when it is combined with said first therapeutically active substance, which are approximately two to ten times lower than the doses required when said second therapeutically active substance is used alone.

The result is to offer a treatment which has the following advantages:
effective at very low doses against susceptible microorganisms,
effective against microorganisms resistant to a therapeutic agent,
effective against microorganisms resistant to several therapeutic agents,
control of recurrence phenomena,
control of phenomena of resistant microorganisms selection.

In all these cases, there is a notable reduction in the risks of toxicity and/or adverse effects well known to the person of the art, thanks to the potentiation which enables the administration of very low doses.

In addition, the costs of producing the treatment are reduced due to the use of small quantities of active substances.

The pharmaceutical compositions according to the invention can be in the form of liposomes or associated with supports such as cyclodextrins or polyethylene glycols.

The pharmaceutical compositions of the invention are a simple and efficient means to combat the problems related to microbial agents in general which comprise mainly resistance to therapeutic agents and toxicity of the latter resulting from the use of high doses.

In fact, carveol, thymol, eugenol, borneol, carvacrol and the derivatives, mixtures and isomers thereof, are simple molecules which have never been described as having any toxicity whatsoever and their addition with its potentiating effect on the second therapeutically active substance enables the use of much lower doses of said second therapeutically active substance.

In a first variant, then, the method for treating patients having a fungal infection consists in administering to said patients the dose, determined by the physician, of the pharmaceutical composition of the invention comprising suitable doses of at least one said first therapeutically active substance, combined with suitable doses of at least one said second therapeutically substance, that is, the suitable antifungal agent.

In a second variant, the method for treating patients having a fungal infection consists in sequentially administering to said patients the dose determined by the physician of at least one said first therapeutically active substance, followed by the suitable dose of at least one said second therapeutically active substance, that is, the suitable antifungal agent, or vice versa.

In this regard, the invention proposes a kit comprising at least one first container containing one of said first therapeutically active substances, and at least one second container containing one of said second therapeutically active substances.

Said kit enables health care personnel to prepare on demand either a mixture of suitable doses of the desired first therapeutic substance(s) and of the desired antifungal agent(s) for a simultaneous administration, or to sequentially and separately administer the suitable dose of at least one said first therapeutically active substance, followed by the suitable dose of at least one said second therapeutically active substance, that is, the suitable antifungal agent, or vice versa. However, a mixture for simultaneous use shall be preferred in order to allow the potentiation complex to form and to act immediately after administration to the patient.

The invention shall become clearer in the following examples describing different embodiments, which are given for purposes of illustration and not by way of limitation.

### EXAMPLE 1

### Treatment of Different Candida albicans Strains with Fluconazole Potentiated by Carvacrol (Fluc P)

In Vitro Tests: Determination of Minimal Fungicidal Concentration (MFC) on Different Strains of Candida albicans

The experiment was carried out with several strains of Candida albicans having different susceptibilities isolated in the hospital environment from the mouth, vagina and digestive tract. The antifungal agent used was fluconazole, an azole derivative which is one of the most effective and most widely used antifungals on the market. An antifungal pharmaceutical composition was prepared by mixing fluconazole at different concentrations with carvacrol at a sub-inhibitory concentration of 0.3 g per liter of solution or excipient. This pharmaceutical composition according to the invention was named FLUC-P for potentiated fluconazole. In each case, antifungal activity was determined either with fluconazole alone, or with carvacrol alone, or with the composition of the invention.

Table 1 gives the results of static tests to determine the minimal fungicidal concentration (MFC) in μg/ml.

TABLE 1

| Candida albicans in | Fluconazole | Composition of the | Carvacrol |
|---|---|---|---|
| exponential growth | alone | invention | alone |
| phase | MFC (μg/ml) | MFC (μg/ml) | MFC (μg/ml) |
| Susceptible strain | 0.5 | / | 1000 |
| Moderately resistant | 150 | 10 | 1000 |
| strain | | | |
| Highly resistant strain | >300 | 10 | 1000 |

Table 1 shows that the composition of the invention had notable fungicidal activity on these strains with different susceptibilities, as compared with fluconazole alone or with carvacrol alone.

Indeed, from Table 1, it is noted that the composition of the invention has a remarkable fungicidal activity on those strains with different susceptibilities as compared with fluconazole alone or with carvacrol alone.

In fact, it can be seen that by using a carvacrol concentration of 0.3 mg/ml, which is approximately three times lower than the MFC of carvacrol alone, the fluconazole concentration which produced fungicidal activity was 15 to 30 times lower than the MFC of fluconazole alone.

Kinetic Tests

Kinetic tests were also carried out on a C. albicans strain highly resistant in order to compare the fungicidal activities of fluconazole alone at a concentration of 300 μg/ml, carvacrol alone at 300 μg/ml and the composition of the invention comprising fluconazole 150 μg/ml and carvacrol 300 μg/ml. The number of colony-forming units (CFU) was measured over time.

The results, shown in FIG. 1, clearly indicate that only the composition of the invention had demonstrable activity against this highly resistant strain.

In Vivo Tests

Groups of 15 mice were experimentally infected by intravenous injection of 10,000,000 cells (colony-forming units) of a Candida albicans strain with moderate resistance to fluconazole.

The first group was composed of control mice which were infected and untreated.

The second group was composed of infected mice treated by gavage, 24 h post-infection, with fluconazole alone at a dose of 4 mg/kg of body weight/day.

The third group was composed of infected mice treated by gavage, 24 h post-infection, with carvacrol alone at a dose of 30 mg/kg of body weight/day.

The fourth group was composed of infected mice treated by gavage, 24 h post-infection, with fluconazole 2 mg/kg of body weight and with carvacrol 30 mg/kg of body weight.

The duration of treatment was seven days for surviving animals. The survival rate was measured over time. The results are given in FIG. 2, which shows that only the mice treated with the pharmaceutical composition were still alive ten days after infection. All the other mice died between the second and seventh day post-infection.

Examination of the organs of mice which died during the experiment (untreated mice and those treated with fluconazole alone or carvacrol alone) revealed high loads of Candida albicans in kidney, lung and bone marrow.

In contrast, in mice treated with the composition of the invention and sacrificed one to ten days after stopping treatment, no C. albicans was found in lung and bone marrow.

In kidney, only two animals still had a very low C. albicans load corresponding to 5% of that seen in the control group. The other animals treated with the composition of the invention had no fungal load in the kidneys.

Here again, it clearly appears that potentiation of fluconazole by carvacrol gives surprising results regarding the reduction in the minimal fungicidal concentration and the rapidity of action in vitro.

This potentiation was also found in vivo in a model of systemic infection.

Now, systemic infection is one of the most serious and life-threatening forms of infection and the most difficult to treat, especially in case of relapse with increasingly resistant microbes.

Other data showed that the composition of the invention comprising fluconazole and carvacrol produced surprising therapeutic effects at doses which were at least two-fold lower than the doses required to treat localized experimental infections (vaginal and oral) in rats and mice.

### EXAMPLE 2

### Treatment of Different Candida albicans Strains with Voriconazole Potentiated by Carvacrol (Vorico-P)

The experiment was carried out with several strains of Candida albicans having different susceptibilities isolated in the hospital environment. The antifungal agent used was voriconazole, an azole derivative and one of the most recent antifungals on the market. An antifungal pharmaceutical composition according to the invention was prepared by mixing voriconazole at different concentrations with carvacrol at a sub-inhibitory concentration of 0.3 g per liter of solution or excipient. This pharmaceutical composition according to the invention was named Vorico-P for potentiated voriconazole. In each case, antifungal activity was determined either with voriconazole alone, or with carvacrol alone, or with the composition of the invention.

Table 2 gives the results of static tests to determine the minimal inhibitory concentration (MIC) and the minimal fungicidal concentration (MFC) in μg/ml.

TABLE 2

| Candida albicans in | | | |
|---|---|---|---|
| exponential growth | Voriconazole alone | Vorico-P | Carvacrol alone |
| phase | MIC (μg/ml) | MFC (μg/ml) | MFC (μg/ml) |
| Susceptible strain | <0.05 | / | 1000 |
| Resistant strain | 10 | <1 | 1000 |

Table 2 shows that the composition of the invention had notable fungicidal activity on the voriconazole-resistant strain, as compared with voriconazole alone or with carvacrol alone.

In fact, it can be seen in Table 2 that by using a carvacrol concentration of 0.3 mg/ml, which is 3.3 times lower than the MFC of carvacrol alone, the voriconazole concentration producing fungicidal activity against resistant strains was at least ten times lower than the concentration of voriconazole alone capable of exhibiting fungistatic activity.

Thus, it can be seen that the potentiation of voriconazole by carvacrol not only allowed a large reduction in the voriconazole dose but also transformed its fungistatic activity into a fungicidal action.

### EXAMPLE 3

### Treatment of Different Candida albicans Strains with Capsofungin Potentiated by Carvacrol (Caspo-P)

The experiment was carried out with several strains of Candida albicans having different susceptibilities isolated in the hospital environment. The antifungal agent used was caspofungin, from the echinocandin family, which is one of the newest antifungals on the market. An antifungal pharmaceutical composition according to the invention was prepared by mixing caspofungin at different concentrations with carvacrol at a sub-inhibitory concentration of 0.3 g per liter of solution or excipient. This pharmaceutical composition according to the invention was named Caspo-P for potentiated caspofungin. In each case, antifungal activity was determined either with caspofungin alone, or with carvacrol alone, or with the composition of the invention.

Table 3 gives the results of static tests to determine the minimal inhibitory concentration (MIC) and the minimal fungicidal concentration (MFC) in μg/ml.

TABLE 3

| Candida albicans in | | | |
|---|---|---|---|
| exponential growth | Caspofungin alone | Caspo-P | Carvacrol alone |
| phase | MIC (μg/ml) | MFC (μg/ml) | MFC (μg/ml) |
| Susceptible strain | <0.05 | / | 1000 |
| Resistant strain | 3 | <0.5 | 1000 |

Table 3 shows that the composition of the invention had notable fungicidal activity on the resistant strain, as compared with caspofungin alone or with carvacrol alone.

In fact, it can be seen in Table 3 that by using a carvacrol concentration of 0.3 mg/ml, which is 3.3 times lower than the MFC of carvacrol alone, the caspofungin concentration producing fungicidal activity was at least six times lower than the concentration of caspofungin alone capable of exhibiting fungistatic activity.

Thus, it can be seen that the potentiation of caspofungin by carvacrol not only allowed a large reduction in the caspofungin dose but also transformed its fungistatic activity into a fungicidal action.

### EXAMPLE 4

### Treatment of Different Candida albicans Strains with 5-Fluorocytosine Potentiated by Euqenol (Fluoro-P)

The experiment was carried out with several strains of Candida albicans having different susceptibilities isolated in the hospital environment. The antifungal agent used was 5-fluorocytosine, from the pyrimidine group, which is one of the oldest antifungal agents. An antifungal pharmaceutical composition according to the invention was prepared by mixing 5-fluorocytosine at different concentrations with eugenol at a sub-inhibitory concentration of 0.5 g per liter of solution or excipient. This pharmaceutical composition according to the invention was named Fluoro-P for potentiated 5-fluorocytosine. In each case, antifungal activity was determined either with 5-fluorocytosine alone, or with eugenol alone, or with the composition of the invention.

Table 4 gives the results of static tests to determine the minimal inhibitory concentration (MIC) and the minimal fungicidal concentration (MFC) in μg/ml.

TABLE 4

| Candida albicans in | 5-fluorocytosine | | |
|---|---|---|---|
| exponential growth | alone | Fluoro-P | Eugenol alone |
| phase | MIC (μg/ml) | MFC (μg/ml) | MFC (μg/ml) |
| Susceptible strain | 2.5 | / | 2000 |
| Resistant strain | 25 | 5 | 2000 |

Table 4 shows that the composition of the invention had notable fungicidal activity on the resistant strain, as compared with 5-fluorocytosine alone or with eugenol alone.

In fact, it can be seen in Table 4 that by using a eugenol concentration of 0.5 mg/ml, which is four times lower than the MFC of eugenol alone, the 5-fluorocytosine concentration producing fungicidal activity on resistant strains was five times lower than the concentration of 5-fluorocytosine alone capable of exhibiting fungistatic activity.

Thus, it can be seen that the potentiation of 5-fluorocytosine by eugenol not only allowed a large reduction in the 5-fluorocytosine dose but also transformed its fungistatic activity into a fungicidal action.

### EXAMPLE 5

### Enlargement of the Spectrum of Action of Fluconazole Potentiated by Carvacrol to Different Aspergillus niger Strains

The experiment was carried out with several strains of Aspergillus niger which were not susceptible to fluconazole. The antifungal agent used was fluconazole, an azole derivative and one of the most widely used antifungals. The biggest drawback of fluconazole is that it does not act on infections due to filamentous fungi. Aspergillosis, caused by microorganisms from the family Aspergillus sp, represents the most common and most difficult-to-treat infection. An antifungal pharmaceutical composition according to the invention was prepared by mixing fluconazole at different concentrations with carvacrol at a sub-inhibitory concentration of 0.25 g per liter of solution or excipient. This pharmaceutical composition according to the invention was named Fluc-P for potentiated fluconazole. In each case, antifungal activity was determined either with fluconazole alone, or with carvacrol alone, or with the composition of the invention.

Table 5 gives the results of static tests to determine the minimal inhibitory concentration (MIC) and the minimal fungicidal concentration (MFC) in μg/ml.

TABLE 5

| Candida albicans in | | | |
|---|---|---|---|
| exponential growth | Fluconazole alone | Fluc-P | Carvacrol alone |
| phase | MIC (μg/ml) | MFC (μg/ml) | MFC (μg/ml) |
| 12 different strains of | >1000 | 150 | 500 |
| Aspergillus niger | | | |

Table 5 shows that the composition of the invention had notable fungicidal activity on Aspergillus niger, as compared with fluconazole alone or with carvacrol alone.

In fact, it can be seen in Table 5 that the use of a carvacrol concentration of 0.25 mg/ml, which is two times lower than the MFC of carvacrol alone, combined with fluconazole at a concentration of 150 μg/ml, led to an outstanding fungicidal activity that fluconazole alone was unable to provide even at a concentration of 1000 μg/ml.

Thus, it can be seen that the potentiation of fluconazole by carvacrol not only allowed a large reduction in the fluconazole dose with species normally treated with fluconazole, but also enlarged the spectrum thereof to filamentous fungi from the Aspergillus genus which are normally not susceptible to fluconazole.

The method for treating a fungal infection consists in administering simultaneously or sequentially to a patient having a fungal infection, the dose determined by the physician of at least one first therapeutically active substance selected from the group consisting of carveol, thymol, eugenol, borneol, carvacrol, and the isomers and derivatives and mixtures thereof, and the determined dose of at least one second therapeutically active substance which is an antifungal agent.

Generally, one simultaneously or sequentially administers to a patient having a fungal infection between 1 and 3000 mg/kg of body weight/day of at least one first therapeutically active substance selected from the group consisting of carveol, thymol, eugenol, borneol, carvacrol, alpha-ionone, beta-ionone and the isomers and derivatives and mixtures thereof, and between 1 and 20 mg/kg of body weight/day of at least one second therapeutically active substance which is an antifungal agent.

In fact, the use of an amount less than 1 mg/kg of body weight/day of said first therapeutically active substance, in particular carvacrol, does not produce the desired potentiation effect.

On the other hand, the use of an amount greater than 3000 mg/kg of body weight/day of said first therapeutically active substance, in particular carvacrol, does not further increase the potentiation effect and poses a greater risk of toxicity.

Likewise, the use of an amount less than 1 mg/kg of body weight/day of said second therapeutically active substance which is an antifungal agent, in particular fluconazole, does not produce the desired therapeutic effect and the use of an amount greater than 20 mg/kg of body weight/day, in particular of fluconazole, does not improve the therapeutic effect and increases the risk of toxicity.

Thus, in a preferred manner, one simultaneously or sequentially administers to a patient having a fungal infection 30 mg/kg of body weight/day of at least one first therapeutically active substance selected from the group consisting of carveol, thymol, eugenol, borneol, carvacrol, alpha-ionone, beta-ionone and the isomers and derivatives and mixtures thereof, and 2 mg/kg of body weight/day of at least one second therapeutically active substance which is an antifungal agent.

More particularly, in the case of Candida albicans infection, one simultaneously or sequentially administers to the patient:
30 mg/kg of body weight/day of carvacrol, and
2 mg/kg of body weight/day of fluconazole.

Of course, the invention is in no way restricted to the embodiments described and illustrated herein which are given solely by way of example.

On the contrary, the invention comprises all the technical equivalents of the methods described herein as well as the combinations thereof where such are carried out in the spirit of the invention.

1-12. (canceled)

13. A pharmaceutical composition comprising:
at least one first therapeutically active substance selected from the group consisting of carveol, thymol, eugenol, borneol, carvacrol, alpha-ionone, beta-ionone, and isomers, derivatives, or mixtures thereof; and
at least one second therapeutically active substance which is an antifungal agent selected from the group consisting of pyrimidines, azoles, echinocandins and mixtures thereof.

14. The composition according to claim 13, wherein the antifungal agent is selected from 5-fluorocytosine, fluconazole, voriconazole, itraconazole, caspofungin or derivatives and mixtures thereof.

15. The composition according to claim 13, wherein said first therapeutically active substance is eugenol or carvacrol and said second therapeutically active substance is 5-fluorocytosine, fluconazole, voriconazole, itraconazole caspofungin or derivatives and mixtures thereof.

16. The composition according to claim 15, wherein said first therapeutically active substance is carvacrol and said second therapeutically active substance is selected from fluconazole, voriconazole, caspofungin or derivatives and mixtures thereof.

17. The composition according to claim 16, wherein said second therapeutically active substance is fluconazole.

18. The composition according to claim 15, wherein said first therapeutically active substance is eugenol and said second therapeutically active substance is 5-fluorocytosine.

19. The composition according to claim 16, wherein said first and second therapeutically active substances are suspended in an aqueous agar solution.

20. The composition according to claim 18, wherein said first and second therapeutically active substances are suspended in an aqueous agar solution.

21. The composition according to claim 16, wherein said composition does not include any detergent or solvent.

22. The composition according to claim 18, wherein said composition does not include any detergent or solvent.

23. A kit comprising:
at least one first container containing a first therapeutically active substance selected from the group consisting of carveol, thymol, eugenol, borneol, carvacrol, alpha-ionone, beta-ionone and isomers, derivatives or mixtures thereof, and
at least one second container containing a second therapeutically active substance which is an antifungal agent selected from the group consisting of pyrimidines, azoles, echinocandins and mixtures thereof.

24. The kit according to claim 23, wherein said second therapeutically active substance is selected from 5-fluorocytosine, fluconazole, voriconazole, itraconazole, caspofungin or derivatives and mixtures thereof.

25. The kit according to claim 23, wherein said first therapeutically active substance is eugenol or carvacrol and wherein said second therapeutically active substance is selected from 5-fluorocytosine, fluconazole, voriconazole, itraconazole, caspofungin or derivatives and mixtures thereof.

26. The kit according to claim 23, wherein said first therapeutically active substance is carvacrol and said second therapeutically active substance is selected from fluconazole, voriconazole, caspofungin, or derivatives and mixtures thereof.

27. The kit according to claim 24, wherein said second therapeutically active substance is fluconazole.

28. The kit according to claim 25, wherein said first therapeutically active substance is eugenol and said second therapeutically active substance is 5-fluorocytosine.

29. A method for treating an infection caused by a fungus comprising the simultaneous or sequential administration to a patient having a fungal infection of:
at least one first therapeutically active substance selected from the group consisting of carveol, thymol, eugenol, borneol, carvacrol, alpha-ionone, beta-ionone and isomers, derivatives, or mixtures thereof, and
at least one second therapeutically active substance which is an antifungal agent, selected from the group consisting of pyrimidines, azoles, echinocandins, and mixtures thereof.

30. The method according to claim 29, wherein said second therapeutically active substance is selected from 5-fluorocytosine, fluconazole, voriconazole, itraconazole, caspofungin, or derivatives and mixtures thereof.

31. The method according to claim 29, wherein said first therapeutically active substance is eugenol or carvacrol and said second therapeutically active substance is selected from 5-fluorocytosine, fluconazole, voriconazole, itraconazole, caspofungin, or derivatives and mixtures thereof.

32. The method according to claim 31, wherein said first therapeutically active substance is carvacrol and said second therapeutically active substance is selected from fluconazole, voriconazole, caspofungin, or derivatives and mixtures thereof.

33. The method of claim 30, wherein said second therapeutically active substance is flavozole.

34. The method according to claim 31, wherein said first therapeutically active substance is eugenol and the second therapeutically active substance is 5-fluorocytosine.

35. The method according to claim 29, wherein:
between 10 and 200 mg/kg of body weight/day of said first therapeutically active substance; and
between 2 and 100 mg/kg of body weight/day of said second therapeutically active substance is simultaneously or sequentially administered to a patient having a fungal infection.
